(19) United States
(12) Patent Application Publication
(10) Pub. No.: US 2016/0264497 A1
(43) Pub. Date: Sep. 15, 2016

(54) PROCESS FOR THE CONVERSION OF METHANE TO C2+ HYDROCARBONS

(71) Applicant: SAUDI BASIC INDUSTRIES CORPORATION, Riyadh (SA)

(72) Inventors: Aghaddin Kh. Mammadov, Sugar Land, TX (US); David West, Bellaire, TX (US)

(21) Appl. No.: 15/035,970

(22) PCT Filed: Nov. 6, 2014
(86) PCT No.: PCT/US2014/064291

§ 371 Date: May 11, 2016

Related U.S. Application Data
(60) Provisional application No. 61/902,546, filed on Nov. 11, 2013.

(51) Int. Cl. C07C 17/10; C07C 2/76

(52) U.S. Cl. CPC C07C 17/10; C07C 2/76; C07C 2523/34; C07C 2523/14; C07C 2523/72; C07C 2523/745; C07C 2523/26; C07C 2523/08; C07C 2523/18; C07C 2523/04

(57) ABSTRACT

A method of making ethane can comprise: introducing a chlorine stream and a methane stream to a first reaction zone comprising a metal oxide catalyst; converting the metal oxide to metal chloride and reacting the methane to form a product stream comprising the C₂H₄; introducing the metal chloride to a second reaction zone and introducing oxygen to convert the metal chloride to metal oxide and chlorine gas; and directing the metal oxide back to the first reaction zone. Also, a method of making ethane, comprising: introducing only a chlorine stream and a methane stream to a metal oxide catalyst to form the ethane and carbon dioxide, wherein the chlorine stream and the methane stream each comprise less than 1 vol % oxygen, and wherein metal oxide converts to metal chloride; and separately converting the metal chloride back to metal oxide with oxygen from an oxygen source, wherein chlorine gas is produced.

### BACKGROUND

Olefins, such as ethylene and propylene are major feedstocks in the organic chemical and petrochemical industries and current feedstocks for the production of ethylene are in relatively short supply. Due to the high demands for ethylene and due to the abundance of natural gas, methods to convert methane to ethylene have been developed.

Currently, there are several methods of converting methane to C₂₊ hydrocarbons. One such conversion reaction occurs by pyrolyzing the methane at high temperatures, for example, greater than 1000 degrees Celsius (° C.), with oxygen to produce ethylene and water. While this method produces ethylene, the produced ethylene in the presence of the oxygen is easily combusted to produce carbon dioxide and water. Catalyzed pyrolysis methods were developed, where a catalyst was used to facilitate the methane conversion reaction. In this method an oxygen feed is required to regenerate said catalyst and high combustion is still observed.

A method was therefore developed such that the methane conversion reaction in the presence of a catalyst could occur without any added oxygen to reduce the likelihood of combustion. In this method, hydrocarbon production in the presence of a catalyst occurs in a physically separate contact zone from an oxygen contact zone, where the catalyst is regenerated. It was found though that the activity of the catalyst was very high and a high amount of the combustion products were still observed in the product stream. Accordingly, this process resulted in a low selectivity for C₂₊ hydrocarbons (i.e. those comprising two or more carbon atoms) of less than 70%.

Improved methods of producing C₂₊ hydrocarbons from methane are therefore desired.

### BRIEF DESCRIPTION

Disclosed herein is a method for making ethane.

In an embodiment: a method of making ethane, comprises: introducing a chlorine stream and a methane stream to a first reaction zone comprising a metal oxide catalyst; converting the metal oxide to metal chloride and reacting the methane to form a product stream comprising the C₂H₄ and CO_{2;} introducing the metal chloride to a second reaction zone; introducing oxygen to the second reaction zone to convert the metal chloride to metal oxide and chlorine gas; and directing the metal oxide back to the first reaction zone.

In another embodiment: a method of making ethane, can comprise introducing only a chlorine stream and a methane stream to a metal oxide catalyst to form the ethane and carbon dioxide, wherein the chlorine stream and the methane stream each comprise less than 1 vol % oxygen, and wherein metal oxide converts to metal chloride; and separately converting the metal chloride back to metal oxide with oxygen from an oxygen source, wherein chlorine gas is produced.

The above described and other features are exemplified by the following figures and detailed description.

### BRIEF DESCRIPTION OF THE DRAWINGS

Refer now to the figures, which are exemplary embodiments, and wherein the like elements are numbered alike.

FIG. 1 is an illustration of a process for the conversion of methane to C₂₊ hydrocarbons; and

FIG. 2 is an illustration of a single location process for the conversion of methane to C₂₊ hydrocarbons.

### DETAILED DESCRIPTION

Current processes for converting methane to C₂₊ hydrocarbons generally display low conversion and/or selectivity for C₂₊ hydrocarbons. The inventors herein developed a process for converting methane to C₂₊ hydrocarbons with a selectivity for C₂ products of greater than or equal to 70%, preferably, greater than or equal to 75%. The current process comprises a first reaction zone, where the methane conversion reaction occurs 1) without the addition of an oxygen (O₂) stream and 2) in the presence of a metal oxide catalyst and chlorine. Not to be bound by theory, the absence of an oxygen stream helps to reduce the amount of combustion that occurs due to added oxygen in the reaction zone and the presence of the chlorine acts to reduce the activity of the catalyst with the hydrocarbons, which reduces combustion due to oxygen in the catalyst. Preferably, chlorine has a higher rate of reaction with the metal oxide catalyst than hydrocarbons and an oxide-metal-chloride phase of the catalyst is formed, which then reacts with methane to form C₂₊ hydrocarbons including ethylene. An example of a methane conversion reaction that occurs in the first reaction zone is reaction 1.

MeO₂+Cl₂+2CH₄→C₂H₄+MeCl₂+2H₂O 1

Reaction 1 shows that the oxygen for the methane conversion reaction originates from the metal oxide catalyst and that the chlorine acts as a reducing agent for the metal oxide catalyst, transforming it to a metal chloride. Accordingly, combustion of the methane in the first reaction zone is reduced and more of the methane is converted into C₂₊ products, preferably, C₂₋₄ products, more preferably, ethane and ethylene. Furthermore, this process has the benefit of converting methane to C₂₊ hydrocarbons with chorine, and without the formation of methyl chloride or hydrogen chloride. It was found that a first product stream from the methane conversion reaction can have less than or equal to 1 vol % of each of methyl chloride and HCl in the first product stream, and preferably is free of methyl chloride and HCl.

In the present method, the metal chloride from reaction 1 is then regenerated in a separate reaction zone, via the following reaction 2.

MeCl₂+O₂→Cl₂+MeO₂ 2

The process comprises adding a methane feed stream, a chlorine feed stream, and regenerated catalyst to a first reaction zone. The methane feed stream can comprise greater than or equal to 40 volume percent (vol %), preferably, greater than or equal to 70 vol %, more preferably, 70 to 100 vol %, even more preferably, 70 to 95 vol % methane based on the total volume of the methane feed stream. The methane feed stream can comprise natural gas. In addition to methane, natural gas can comprise ethane, carbon dioxide, propane, butanes, pentanes, nitrogen, hydrogen sulphide, oxygen, and rare gases (such as argon, helium, neon, and xenon gas). The methane feed stream can comprise less than 0.3 vol %, preferably, 0 to 0.2 vol %, even more preferably, 0 vol % oxygen. The chlorine stream can comprise HCl, Cl₂, or a combination comprising one or both of the foregoing. The chlorine stream can comprise recycled chlorine obtained, for example, from the second reaction zone. Likewise, the methane feed and the chlorine feed can be premixed and can enter the first reaction zone as a single stream. The elemental chlorine (Cl) can be fed into the reactor at 1 to 5 vol %, preferably, 1 to 3 vol % based on the combined volumes of the methane feed and the chlorine feed. Accordingly, if the chlorine feed comprises Cl₂, then the Cl₂ can be fed into the reactor at 0.5 to 2.5 vol %, preferably, 0.5 to 1.5 vol % based on the combined volumes of the methane feed and the chlorine feed. The chlorine feed can initially comprise fresh HCl and can later comprise recycled Cl₂ from the second reaction zone.

The first reaction zone can be a separate reactor from the second reaction zone. In such case, the first reaction zone reactor and the second reaction zone reactor can each independently be, for example, a fluidized bed reactor, an ebullating be reactor, or an entrained bed reactor. The first reaction zone and the second reaction zone can be located in two different locations in a single reactor, where, in such a set-up, heat only needs to be applied to one reactor. The first reaction zone and the second reaction zone can be located in the same location, where the location acts as a first reaction zone for an amount of time and then acts as a second reaction zone for an amount of time.

The catalyst can comprise a metal oxide (MeOx) that is capable of catalyzing the methane conversion reaction. The metal can be a redox element which can carry the redox cycles between the two reaction zones. Preferably, the metal can comprise manganese (Mn), tin (Sn), lead (Pb), copper (Cu), iron (Fe), chromium (Cr), indium (In), germanium (Ge), antimony (Sb), bismuth (Bi), or a combination comprising one or more of the foregoing, preferably the metal comprises germanium. The catalyst can be a supported catalyst. Examples of support material are MgO, SiO₂, Al₂O₃, and ZrO₂. When a support is used, the metal oxide can be present in an amount of 1 to 50 wt % based on the total weight of the catalyst. The catalyst can further comprise an alkali metal such as sodium (Na), potassium (K), magnesium (Mg), calcium (Ca), or a combination comprising one or more of the foregoing. The catalyst can be for example, a sodium manganese catalyst, such as Na_{0.7}MnO₂ that is supported on a silica gel.

The methane conversion reaction can occur in the first reaction zone and can occur at a pressure of 2 to 100 atmospheres (atm), preferably, 3 to 30 atm. The methane conversion reaction can occur at a temperature of 500 to 1000° C., preferably, 750 to 800° C. The methane conversion reaction can occur at a temperature of 450 to 800° C., preferably, 450 to 750° C., more preferably, 500 to 600° C. The feed entering the first reaction zone (including the methane feed stream, the chlorine feed stream, and the regenerated catalyst stream) can have less than 1 vol % 0₂, preferably, less than or equal to 0.5 vol % 0₂, and more preferably less than or equal to 0.3 vol % 0₂, and especially preferred is if the feed to the first reaction zone is free of 0₂. Desirably, each of the methane feed stream, the chlorine feed stream, and the regenerated catalyst stream, independently, comprise less than or equal 0.3 vol %, preferably, 0 to 0.2 vol %, more preferably, 0 vol % of 0₂.

The residence time of the catalyst in the first reaction zone can vary and can depend on, for example, the specific catalyst used, the concentration and feedrate of the methane in the methane feed stream, and the temperature and pressure in the first reaction zone. The residence time of the catalyst in the first reaction zone can be 0.04 to 30 seconds (sec), preferably, 0.4 to 1 sec, where the residence time is the amount of time the feed is in contact with the catalyst. The residence time of the methane feed with the catalyst can vary based on the processing conditions. The contact time can be 0.1 to 10 seconds (s), preferably, 1 to 5 seconds.

A first product stream can exit the first reaction zone. The first product stream can comprise unreacted methane, ethane, ethylene, carbon dioxide, propane, propene, butane, butene, or a combination comprising one or more of the foregoing. The first product stream can comprise 3 to 7 vol % of ethylene. Any unreacted methane in the first product stream can be recovered and recycled back into the first reaction zone.

In the first reaction zone, the metal oxide catalyst can be converted to spent catalyst in the form of a metal chloride. The spent catalyst can be removed from the first reaction zone and introduced to a second reaction zone. Oxygen can also be introduced to the second reaction zone, for example, as pure oxygen or as an air feed. Regenerated catalyst produced in the second reaction zone can exit the second reaction zone and can enter the first reaction zone.

The catalyst regeneration can occur at a temperature of 300 to 1200° C., preferably, 450 to 800° C., more preferably, 450 to 750° C., even more preferably, 550 to 650° C. The catalyst regeneration can occur at a pressure of less than or equal to 30 atm. The temperature in the first and second reaction zones can be the same. The catalyst can be in the catalyst regeneration zone (e.g., in the second reaction zone) for 0.3 to 12 sec. Preferably, the catalyst can be in the regeneration zone and can be in contact with a sufficient amount of oxygen to oxidize greater than 90 wt %, preferably, 90 to 100 wt % of the catalyst to the fully oxidized metal oxide form and to combust (preferably completely combust) any carbonaceous deposit material deposited on the catalyst.

Chlorine can leave the second reaction zone. When the oxygen feed to the second reaction zone comprises a gas other than oxygen, said gas can also exit the second reaction zone. For example, if the oxygen feed is air, then nitrogen can also exit the second reaction zone. By regenerating the catalyst in a second reaction zone, the oxygen can be added only in the second reaction zone and the first reaction zone can remain free of added oxygen, thereby reducing methane combustion. Also, as the oxygen is only in the second reaction zone, the oxygen does not need to be high purity oxygen and can be added as air. In co-feed processes where oxygen is added with the methane, air cannot generally be used as it is difficult to separate the nitrogen from the product stream. In the present process, it is easy to separate out any nitrogen from the chlorine in the second product stream.

Ethylene from the first product stream and chlorine from the second product stream can go to a third reactor to form vinyl chloride.

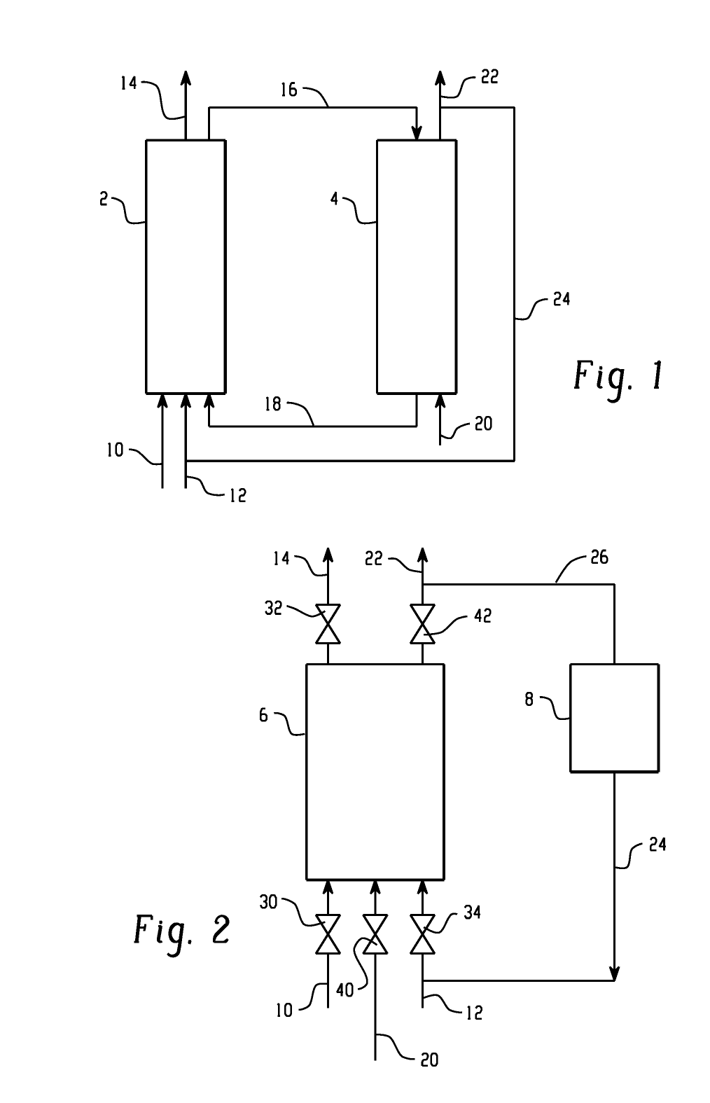

FIG. 1 illustrates a process for the conversion of methane to C₂₊ hydrocarbons. FIG. 1 shows methane feed stream 10, chlorine feed stream 12, and regenerated catalyst stream 18 entering first reaction zone 2. While the figure illustrates the three streams as separate streams, it is understood that two or more of said streams can be combined before entering the first reaction zone 2. The methane conversion reactor occurs in the first reaction zone to produce at least ethylene and carbon dioxide that exits the first reaction zone 2 as first product stream 14. The ethylene and/or carbon dioxide can be separated from the first product stream 14 and the ethylene can be used for example in a reaction to make vinyl chloride.

During the methane conversion reaction in the first reaction zone 2, the activity of the metal oxide catalyst decreases and the metal oxide is converted to metal chloride. The metal chloride exits the first reaction zone 2 as spent catalyst stream 16. Spent catalyst stream 16 is introduced to second reaction zone 4 where it is regenerated via the introduction of oxygen feed stream 20. Regenerated catalyst stream 18 then exits the second reaction zone 4 and is reintroduced to the first reaction zone 2 via regenerated catalyst stream 18. The chlorine (Cl₂) by product of the regeneration reaction and any other gas, for example nitrogen if air is used as the oxygen feed stream 20, exits the second reaction zone 4 as second product stream 22. The Cl₂ can be separated from the second product stream 22. All or a portion of the Cl₂ from the second product stream 22 can be used, for example, in a reaction with ethylene to make vinyl chloride and/or as recycled chlorine that can enter the first reaction zone 2 as recycle chlorine stream 24.

Likewise, the disclosed process can occur in a single reactor, where the methane conversion reaction and the regeneration reaction occur in the same location. When the process occurs in a single location, the catalyst is not cycled from a first location to a second location and instead, the single location cycles between being the first reaction zone and the second reaction zone by controlling the feed stream to the single location.

FIG. 2 illustrates a process for methane conversion wherein the first and second reaction zones occur in the same location, but at different times. Accordingly, FIG. 2 shows methane feed stream 10 and chlorine feed stream 12 entering the reactor 6. At this time, valves 30, 32, and 34 are open and valves 40 and 42 are closed. Valve 42 is closed and the reaction products, including any ethylene produced during the methane conversion phase, leave the reactor 6 as first product stream 14.

After an amount of time, where the catalyst activity is reduced, for example as determined by ethylene concentration in the first product stream 14, the flow of methane and chlorine to the reactor 6 is stopped by closing valves 30 and 34, respectively. The regeneration phase is then started by further closing valve 32 and opening valve 40 to let the oxygen enter the reactor 6 via oxygen feed stream 20 and opening valve 42 to let the second product stream 22 exit the reactor 6. All or a portion of the chlorine exiting in second product stream 22 can be recycled by directing the chlorine via chlorine stream 26 to chlorine storage tank 8. Stored chlorine from the chlorine storage tank 8 can later be used in the chlorine feed stream 12 during the methane conversion phase of the process.

It is noted that while FIG. 2 illustrates streams 10 and 12 as separate feed streams, these two streams can be combined to be one stream before entering the reactor 6. Likewise, it is noted that while FIG. 2 illustrates streams 14 and 22 as separate streams exiting the reactor 6, one can envision one stream leaving the reactor 6 where the composition leaving the reactor is dependent upon the reaction occurring at any given time.

Set forth below are some embodiments of the present method of making ethane.

Embodiment 1: a method of making ethane, comprising: introducing a chlorine stream and a methane stream to a first reaction zone comprising a metal oxide catalyst; converting the metal oxide to metal chloride and reacting the methane to form a product stream comprising the C₂₊ hydrocarbon and CO₂, wherein the C₂₊ hydrocarbon comprises at least one of ethane and ethene; introducing the metal chloride to a second reaction zone; introducing oxygen to the second reaction zone to convert the metal chloride to metal oxide and chlorine gas; and directing the metal oxide back to the first reaction zone.

Embodiment 2: a method of making ethane, comprising: introducing only a chlorine stream and a methane stream to a metal oxide catalyst to form the ethane and carbon dioxide, wherein the chlorine stream and the methane stream each comprise less than 1 vol % oxygen, and wherein metal oxide converts to metal chloride; and separately converting the metal chloride back to metal oxide with oxygen from an oxygen source, wherein chlorine gas is produced.

Embodiment 3: the method of any of Embodiments 1-2, wherein the chlorine stream and the methane streams are introduced to the metal oxide catalyst as a mixed stream.

Embodiment 4: the method of any of Embodiments 1-3, wherein the oxygen source is air.

Embodiment 5: the method of Embodiment 4, further comprising separating nitrogen from the chloride.

Embodiment 6: the method of any of Embodiments 1-5, further comprising recycling the chlorine gas to the first reaction zone.

Embodiment 7: the method of any of Embodiments 1-6, wherein no oxygen gas is fed to the first reaction zone.

Embodiment 8: the method of any of Embodiments 1-7, wherein the chlorine stream comprises hydrogen chloride.

Embodiment 9: the method of any of Embodiments 1-8, further comprising recycling at least a portion of the chlorine gas to the first reaction zone.

Embodiment 10: the method of any of Embodiments 1-9, wherein the chlorine is fed to the first reaction zone in an amount of 1-5 vol % based upon the total volume of the methane stream and the chlorine stream.

Embodiment 11: the method of any of Embodiments 1-10, wherein selectivity for ethene is greater than or equal to 70%.

Embodiment 12: the method of any of Embodiments 1-11, wherein metal comprises manganese (Mn), tin (Sn), lead (Pb), copper (Cu), iron (Fe), chromium (Cr), indium (In), germanium (Ge), antimony (Sb), bismuth (Bi), or a combination comprising one or more of the foregoing.

Embodiment 13: the method of any of Embodiments 1-12, wherein the metal oxide catalyst is modified with an alkali metal comprising sodium (Na), potassium (K), magnesium (Mg), calcium (Ca), or a combination comprising one or more of the foregoing.

Embodiment 14: The method of any of Embodiments 1-13, wherein less than or equal to 1 vol % O₂ is introduced to the first reaction zone, based upon a total volume of feed introduced to the first reaction zone.

Embodiment 15: The method of any of Embodiments 1-14, wherein each of the methane stream, the chlorine stream, and any recycled chlorine stream, independently, comprise less than or equal 0.3 vol % O₂, based upon a total volume of all feed streams.

Embodiment 16: The method of any of claims 1-15, wherein each of the methane stream, the chlorine stream, and any recycled chlorine stream, independently, comprise less than or equal 0.2 vol % O₂, based upon a total volume of all feed streams.

Embodiment 17: a method of making vinyl chloride monomer (VCM), comprising: forming the ethene and the chlorine gas according to any of Embodiments 1-16; reacting at least a portion of the chlorine gas and the ethene to form the vinyl chloride monomer.

Embodiment 18: the method of Embodiment 17, wherein the oxygen source is air, and further comprising, prior to reacting the ethene and chlorine gas, separating N₂ from the chlorine gas.

Embodiment 19: the method of any of Embodiments 17-18, further comprising, prior to reacting the C₂H₄ and chlorine gas, separating the CO₂ from the C₂H₄.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

All ranges disclosed herein are inclusive of the endpoints, and the endpoints are independently combinable with each other (e.g., ranges of “up to 25 wt %, or, more preferably, 5 to 20 wt %,” is inclusive of the endpoints and all intermediate values of the ranges of “5 to 25 wt %,” etc.). “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment,” “another embodiment,” “an embodiment,” and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments. Disclosure of a narrower range in addition to a broader range is not a disclaimer of the broader range. This Application claims priority to U.S. patent application 61/902,546 filed Nov. 11, 2013.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of making C₂₊ hydrocarbon, comprising:
introducing a chlorine stream and a methane stream to a first reaction zone comprising a metal oxide catalyst;
converting the metal oxide to metal chloride and reacting the methane to form a product stream comprising the C₂₊ hydrocarbon and CO₂, wherein the C₂₊ hydrocarbon comprises at least one of ethane and ethene;
introducing the metal chloride to a second reaction zone;
introducing an oxygen source to the second reaction zone to convert the metal chloride to metal oxide and chlorine gas; and
directing the metal oxide back to the first reaction zone.

2. The method of claim 1, wherein less than or equal to 1 vol % O₂ is introduced to the first reaction zone, based upon a total volume of feed introduced to the first reaction zone.

3. A method of making ethane, comprising:
introducing only a chlorine stream and a methane stream to a metal oxide catalyst to form the ethane and carbon dioxide, wherein the chlorine stream and the methane stream each comprise less than 1 vol % oxygen, and wherein metal oxide converts to metal chloride; and
separately converting the metal chloride back to metal oxide with oxygen from an oxygen source, wherein chlorine gas is produced.

4. The method of claim 1, wherein the chlorine stream and the methane streams are introduced to the metal oxide catalyst as a mixed stream.

5. The method of claim 1, wherein the oxygen source is air.

6. The method of claim 5, further comprising separating nitrogen from the chlorine stream.

7. The method of claim 1, further comprising recycling the chlorine gas to the first reaction zone.

8. The method of claim 1, wherein no oxygen gas is fed to the first reaction zone.

9. The method of claim 1, wherein the chlorine stream comprises hydrogen chloride.

10. The method of claim 1, further comprising recycling at least a portion of the chlorine gas to the first reaction zone.

11. The method of claim 1, wherein the chlorine is fed to the first reaction zone in an amount of 1-5 vol % based upon the total volume of the methane stream and the chlorine stream.

12. The method of claim 1, wherein selectivity for ethene is greater than or equal to 70%.

13. The method of any of claims 1 12 claim 1, wherein each of the methane stream, the chlorine stream, and any recycled chlorine stream, independently, comprise less than or equal 0.3 vol % O₂, based upon a total volume of all feed streams.

14. The method of any of claim 1, wherein the metal oxide catalyst comprises a metal comprises manganese (Mn), tin (Sn), lead (Pb), copper (Cu), iron (Fe), chromium (Cr), indium (In), germanium (Ge), antimony (Sb), bismuth (Bi), or a combination comprising one or more of the foregoing.

15. The method of claim 1, wherein the metal oxide catalyst is modified with an alkali metal comprising sodium (Na), potassium (K), magnesium (Mg), calcium (Ca), or a combination comprising one or more of the foregoing.

16. A method of making vinyl chloride monomer (VCM), comprising:
forming the ethene and the chlorine gas according to claim 1;
reacting at least a portion of the chlorine gas and the C₂H₄ to form the vinyl chloride monomer.

17. The method of claim 16, wherein the oxygen source is air, and further comprising, prior to reacting the ethene and chlorine gas, separating N₂ from the chlorine gas.

18. The method of claim 16, further comprising, prior to reacting the ethene and chlorine gas, separating the CO₂ from the ethene

19. The method of claim 16, wherein each of the methane stream, the chlorine stream, and any recycled chlorine stream, independently, comprise less than or equal 0.3 vol % O₂, based upon a total volume of all feed streams.

20. The method of claim 3, wherein each of the methane stream, the chlorine stream, and any recycled chlorine stream, independently, comprise less than or equal 0.3 vol % O₂, based upon a total volume of all feed streams.
